(19) United States
(12) Patent Application Publication
(10) Pub. No.: US 2018/0098483 A1
(43) Pub. Date: Apr. 12, 2018

(54) Plant Yield Benefits By Microbials

(71) Applicant: Monsanto Technology LLC, St. Louis, MO (US)

(72) Inventors: Bradon James Fabbri, Chesterfield, MO (US); Ken Ferreira, Wentzville, MO (US); Janne Kerovuo, St. Louis, MO (US); Matthew Mccown, Wildwood, MO (US); Radha G. Mohanty, Ballwin, MO (US); Scott R. Schaecher, Webster Groves, MO (US)

(73) Assignee: Monsanto Technology LLC, St. Louis, MO (US)

(21) Appl. No.: 15/568,373

(22) PCT Filed: Apr. 22, 2016
(86) PCT No.: PCT/US16/28983

§ 371 Date: Oct. 20, 2017

Related U.S. Application Data
(60) Provisional application No. 62/151,360, filed on Apr. 22, 2015.

(51) Int. Cl. A01C 1/06; A01N 43/16; A01N 63/02; A01N 63/04; C12R 1/125; C12N 1/14; C12R 1/885; A01H 5/10

(52) U.S. Cl. CPC A01C 1/06; A01N 43/16; A01N 63/02; A01H 5/10; C12R 1/125; C12N 1/14; C12R 1/885; A01N 63/04

(57) ABSTRACT

Microbe combinations, a seed coated with one of the combinations and a plant protected from corn on corn yield penalties by one of the combinations. Further provided as a microbe combination applied to the seed, stalk or leaf that results in an increase in plant yield.

### BACKGROUND

Seeds are often treated with chemical or biological pesticides, such as fungicides, insecticides, and nematicides, including captan, metalaxyl and Maxim, to control fungi, insects, and nematodes.

Trichoderma is a genus of fungi that contains about 20 species. Synonyms for the genus name include Aleurisma and Sporoderma. Trichoderma virens, which is also called Gliocladium virens, is a member of the genus. The natural habitats of these fungi include soil and plant material. A member of the genus, Trichoderma harzianum KRL-AG2 (ATCC 20847) also known as strain T-22, is used as an innoculant that is applied as a seed or soil treatment or on cuttings and transplants. Other known and commercially available Trichoderma virens strains include those having the following ATCC accession numbers: 10043, 10044, 10045, 13213, 13362, 204067, 204443, 204444, 204445, 20903, 20904, 20906, 24290, 42955, 44327, 44734, 48179, 52045, 52199, 58676, 58677, 58678, 62399, 64271, 74180, 9645, MYA-297, MYA-298, MYA-649 and MYA-650.

Bacillus is a genus of rod-shaped, gram-positive, aerobic or (under some conditions) anaerobic bacteria. Bacillus species are widely found in soil. Bacillus amyloliquefaciens is a spore-forming member of the genus. Bacillus amyloliquefaciens L. L. Campbell strain F (ATCC 23350) is one type strain for the species. Other known and commercially available Bacillus amyloliquefaciens strains include those having the following ATCC accession numbers: 23842, 23843, 23844, 23845, 31592, 49763, 53495 and BAA-390 (Int. J. Sys. Bacteriol. 37:69-71, 1987; J. Bacteriol. 94:1124-1130, 1967).

Bacillus amyloliquefaciens is also called Bacillus subtilis var. amyloliquefaciens by some investigators. A protease produced from Bacillus subtilis var. amyloliquefaciens is commonly used as a tenderized for raw meat products. According to the U.S. Environmental Protection Agency (EPA), Bacillus subtilis var. amyloliquefaciens strain FZB24 is a naturally-occurring microorganism and widespread in the environment. Bacillus subtilis var. amyloliquefaciens FZB24 (EPA Registration Number: 72098-5 and EPA Establishment Number: 73386-DEU-001) is known and commercially available at a reasonable price, being marketed under the trademark Taegro®.

Bacillus lentimorbus is another spore-forming member of the genus. Bacillus lentimorbus Dutky 1940 (ATCC 14707) is the type strain for the species (Skerman, V. B. D., McGowan, V., and Sneath, P. H. A., Approved lists of bacterial names. Int. J. Syst. Bacteriol. 30: 225-420, 1980). Some researchers consider Bacillus lentimorbus to be a variety of Bacillus popilliae. Bacillus lentimorbus and Bacillus popilliae have recently been reclassified as Paenibacillus lentimorbus and Paenibacillus popilliae (Pettersson, B., Rippere, K. E., Yousten, A. A. and Priest, F. G., Transfer of Bacillus lentimorbus and Bacillus popilliae to the genus Paenibacillus with emended descriptions of Paenibacillus lentimorbus comb. nov. and Paenibacillus popilliae comb. nov., Int. J. Syst. Bacteriol. 49: 531-540, 1999).

It is known that, when the same crop is planted on the same plot of land in consecutive years, the yield of the crop from the plot of land decreases over time. This is called a “yield penalty.” One solution to this problem is to “rotate” crops. That is, instead of planting the same crop on the same plot of land in consecutive years, different crops are planted on the plot in consecutive years to reduce the yield penalty. Yield penalty, which can occur with a variety of crops, and crop rotation, can be disadvantages to farmers, because they may restrict the type of crop that can be grown on a plot of land at a specific time.

### SUMMARY

This disclosure relates to microbe combinations, a seed or plant coated with said combinations and using the microbial combinations as an inoculant. In particular, the disclosure provides for, and includes microbe combinations and their use for providing for improved yields when planting the same crop in sequential growing seasons. In one example, the disclosed methods for using the microbe combinations may be used to lessen, or even eliminate, the reduced yield that may occur when the same crop is grown on a plot of land in consecutive years. The disclosed methods describe a way to rescue or partially rescue the yield penalty that occurs when the same crop is planted in consecutive years. In one example, the disclosed methods can be used to rescue or partially rescue the yield penalty that occurs when corn is planted on the same land in consecutive years.

The disclosure provides for, and includes, methods to improve yield of plants and plant seed production.

One advantage to the methods disclosed herein is that it provides an inoculant as an effective means of minimizing impact to yield without crop rotation management, i.e. does not require a farmer to plant a second different crop in rotation. A further advantage of the embodiments according the present disclosure is that their use produces more consistent results, as shown by the Working Examples presented herein. In fact, use of the combinations disclosed herein is shown to be functional when use of its individual constituent is not.

The compositions disclosed herein may be used in combination with other crop management systems.

Through a variety of laboratory and field trials that Bacillus subtilis var. amyloliquefaciens TJ 1000 and Trichoderma virens G1-3 it has been shown that such strains are compatible with one another and that they act together to produce increased yield in plants.

The present disclosure provides for, and includes an inoculum, a seed coated with the inoculum, a plant protected with the inoculum, a method of producing the inoculum and a method of protecting a seed or a plant with the inoculum. A further embodiment of the inoculum comprises a combination of a species of Trichoderma and a species of Bacillus, preferably a spore-forming strain of Bacillus. More preferably, the strain may be Trichoderma virens and the other strain is Bacillus subtilis var. amyloliquefaciens, although other combinations are also envisioned. In one embodiment, the strain is Trichoderma virens G1-3 (ATCC 58678) or Trichoderma virens G1-21 (an isolate that is commercially available from Thermo Trilogy Corporation) and the other strain is Bacillus subtilis var. amyloliquefaciens TJ1000 or 1BE (ATCC BAA-390) or Bacillus subtilis var. amyloliquefaciens FZB24 (a strain that is commercially available from Earth Biosciences, Inc.).

Further embodiments of the disclosure comprise combining of a Trichoderma virens fungus and a Bacillus amyloliquefaciens bacterium and placing this combination on a seed or in the vicinity of the seed or seedling. A person having ordinary skill in the art would understand that the names Trichoderma virens and Gliocladium virens are synonymous. The ATCC listing of this organism under ATCC Accession No. 58678 confirms its prior classification as Gliocladium virens.

In a further embodiment, the inoculum is produced by adding an essentially pure culture, a substantially pure culture, an axenic culture or a biologically pure culture of Trichoderma virens to a bioreactor containing molasses-yeast extract growth medium using a standard inoculation technique. The medium is agitated and aerated and its temperature is maintained at about 28° C. After the Trichoderma virens is grown in the medium for about eight hours, an essentially pure culture, a substantially pure culture, an axenic culture or a biologically pure culture of Bacillus amyloliquefaciens is added to the medium using a standard inoculation technique. The combined, competitive culture is grown under the aforementioned conditions and produces maximum cell and spore counts in approximately seven days. The combined culture is then used as an inoculum and is applied each seed at a rate of no less than about 1,000 spore counts per seed.

In a further embodiment, a solution containing an essentially pure culture, a substantially pure culture, an axenic culture or a biologically pure culture of the fungus Trichoderma virens is combined with a solution containing an essentially pure culture, a substantially pure culture, an axenic culture or a biologically pure culture of Bacillus amyloliquefaciens in a 50/50 mixture by volume and is applied to a seed at a rate of no less than about 10,000 spore counts per seed.

In one embodiment, an agricultural inoculum suitable for treating plant seeds comprises a Trichoderma virens fungus selected from the group consisting of isolate ATCC 58678, isolate G1-21 and mutants thereof; a Bacillus subtilis var. amyloliquefaciens bacterium selected from the group consisting of strain ATCC BAA-390, strain FZB24 and mutants thereof, and a suitable carrier that is non-phytotoxic, non-bacteriostatic, and non-bactericidal. Suitable carriers include wettable clay based powders, dextrose granules or powders, sucrose granules or powders and maltose-dextrose granules or powders.

A further embodiment is a composition of matter comprising a plant seed inoculated with a combination comprising a Trichoderma virens antagonist selected from the group consisting of isolate ATCC 58678, isolate G1-21 and mutants thereof and a Bacillus amyloliquefaciens antagonist selected from the group consisting of strain ATCC BAA-390, strain FZB24 and mutants thereof, wherein said combination improves yield.

A further embodiment is a process for making a composition comprising introducing an essentially pure culture of Bacillus amyloliquefaciens (strain FZB24) to a growth medium about eight hours after an essentially pure culture of Trichoderma virens (isolate G1-21) is introduced to the growth medium and growing the culture as a competitive culture.

A further embodiment is a process comprising making a composition by combining an essentially pure culture of Trichoderma virens G1-3 (isolate G1-21) with an essentially pure culture of Bacillus amyloliquefaciens (strain FZB24) in a 50:50 mixture and applying said composition to a seed at a rate of at least 100,000 spores per seed.

In one embodiment, the spore count applied per seed ranges from about 1,000 to about 1,000,000, regardless of seed size. In another embodiment according to the present disclosure, the spore count per seed is from about 1,000 to about 10,000. In a further embodiment, the spore count per seed is from about 10,000 to about 100,000. In a yet further embodiment, the spore count per seed is from about 100,000 to about 1,000,000. In a yet another embodiment provided by the present disclosure, the spore count per seed is from about 1,000,000 to about 2,000,000.

A further embodiment is a method for improving yields of corn and preventing corn on corn yield penalties comprising placing in the growing medium in the immediate vicinity of the plant to be protected an effective quantity of one of the microbe combinations disclosed herein.

Yet a further embodiment is a method for improving yields of corn and reducing corn on corn yield penalties comprising adding one of the microbe combinations disclosed herein in an effective quantity to a substrate such as pelletized calcium sulfate or pelletized lime and placing the pellet in the immediate vicinity of the plant to be protected. The pellet may or may not contain other nutrients.

A further embodiment is a method for improving corn yield comprising adding one of the microbe combinations disclosed herein in an effective quantity to a liquid solution such as water and applying the liquid solution in the immediate vicinity of the plant to be protected.

In a further embodiment, the compositions disclosed herein may or may not contain additional nutrients and may include one or more chemical pesticides applied to the seed such as, for example, ipconazole, metalaxyl, trifloxystrobin, clothiandin, VOTiVO, thiamethoxam, cyantaniliprole, azoxystrobin, fludioxonil, or tioxazafen. The disclosed combination may also be added to a plant nutrient (nitrogen-phosphorus-potassium (NPK)) plus plant micro-nutrient solution that is compatible with the combination and applied as an in-furrow treatment. The disclosed combinations may also be added to biologically active ingredients including, one or more lipo-chitooligosaccarides and derivatives thereof, one or more mycorrhizae (Myc factors) and derivatives thereof, one or more chitinous compounds and derivatives thereof, one or more chitooligosaccharides and derivatives thereof, one or more flavonoids and derivatives thereof, one or more non-flavonoid nod gene inducers and derivatives thereof, one or more karrikins and derivatives thereof, or any signal molecule combination.

A further embodiment is a method comprising inoculating a seed of the plant with an effective amount of a microbial inoculant comprising a combination of microorganisms having all of the identifying characteristics of Trichoderma virens and Bacillus amyloliquefaciens, said inoculation resulting in the enhance availability or uptake of plant nutrients or improved soil characteristics.

Yet a further embodiment involves combining a Trichoderma virens fungus and a Bacillus amyloliquefaciens bacterium to enhance ease of use and longevity of shelf life both as a stored product and when applied to a seed. In a further embodiment, the disclosure provides for, and includes applying the disclosed Trichoderma microorganism and the Bacillus microorganism to a wettable powder, in which form it is applied.

A further embodiment is a composition of matter made by combining: a composition made by combing a plurality of microbes selected from the group consisting of a Trichoderma virens fungus selected from the group consisting of isolate G1-21, selections or mutants thereof and a Bacillus amyloliquefaciens bacterium selected from the group consisting of strain FZB24, selections or mutants thereof; a Trichoderma virens fungus selected from the group consisting of isolate ATCC 58678, selections or mutants thereof and a Bacillus amyloliquefaciens bacterium selected from the group consisting of strain FZB24, selections or mutants thereof; and a Trichoderma virens fungus selected from the group consisting of isolate ATCC 58678 and mutants thereof and a Bacillus amyloliquefaciens bacterium selected from the group consisting of strain FZB24, selections or mutants thereof; and a suitable carrier that is non-phytotoxic, non-bacteriostatic, and non-bactericidal.

A further embodiment is an microbial combination improving year on year plant yields of the same crop made by combining effective amounts of: a fungus selected from the group of Trichoderma virens isolate (isolate G1-21), selections or mutants thereof; a bacterium selected from the group of Bacillus amyloliquefaciens (strain FZB24), selections or mutants thereof; and a suitable carrier that is non-phytotoxic, non-bacteriostatic, and non-bactericidal.

Yet a further embodiment is a seed assembly made by combining a plant seed with effective amounts of a Trichoderma virens fungus and a Bacillus subtilis var. amyloliquefaciens bacterium. In a further embodiment, the seed is a seed of a plant selected from the group of a monocot, and a dicot. In a further embodiment, the seed is a seed of a plant selected from the group of a legume plant, and a non-legume plant. In a further embodiment, the seed is a seed of a plant selected from the group of corn, sunflower, soybean, field pea, and wheat.

Yet a further embodiment is a process comprising: making a composition by combining an essentially pure culture of Trichoderma virens (isolate G1-21) with an essentially pure culture of Bacillus amyloliquefaciens (strain FZB24) in a mixture; and applying said composition to a seed; wherein said mixture ranges in composition from 10 to 90 percent Trichoderma virens (isolate G1-21) by volume and from 90 to 10 percent Bacillus amyloliquefaciens (strain FZB24) by volume.

Yet a further embodiment is a process comprising: making a composition by combining an essentially pure culture of Trichoderma virens (isolate G1-21) with a plurality of essentially pure cultures of bacteria in a mixture; and applying said composition to a seed; wherein said mixture ranges in composition from 10 to 90 percent Trichoderma virens (isolate G1-21) by culture volume.

In one embodiment the mixture ranges in composition from 10 to 90 percent Trichoderma virens by volume and from 90 to 10 percent Bacillus amyloliquefaciens by volume. In another embodiment, the mixture comprises about 20 percent Trichoderma virens by volume 80 percent Bacillus amyloliquefaciens by volume. In a further embodiment, the mixture comprises about 30 percent Trichoderma virens by volume 70 percent Bacillus amyloliquefaciens by volume. In a yet further embodiment, the mixture comprises about 40 percent Trichoderma virens by volume 60 percent Bacillus amyloliquefaciens by volume.

In one embodiment, a method comprises combining a spore-forming fungal strain and a spore-forming bacterial strain to produce a product comprising a composition of matter disclosed herein; and applying the product to a plant or to a part of the plant that is the same as was planted in the year prior; whereby application of the product produces yield enhancement in the plant even with the known corn-on-corn yield penalty.

In another embodiment, the disclosure provides for, and includes a method comprising: applying a Trichoderma spp. microorganism and a Bacillus spp. microorganism to a wettable powder to produce a combination comprising a composition disclosed herein; and applying the combination to a seed; whereby application of the combination produces a positive yield response in a plant growing from the seed, which is the same plant that was grown in the immediately previous growing season.

In yet another embodiment, the composition of matter as disclosed herein ranges in composition from 1 to 99 percent Trichoderma vixens by culture volume and from 99 to 1 percent Bacillus amyloliquefaciens by culture volume.

In another embodiment, the composition of matter comprises: a plant seed inoculated with an agricultural inoculum disclosed herein; wherein said combination increases the yield of the plant. In another embodiment, the disclosure provides for, and includes a method for increasing the yield of a plant, the method comprising: coating a seed of the plant with an effective amount of an agricultural inoculum disclosed herein; and culturing the plant.

In yet another embodiment, disclosure provides for, and includes a composition made by combining effective amounts of: a spore-forming fungus; and a spore-forming bacterium; wherein the spore-forming fungus does not produce a substance that substantially inhibits the growth of the spore-forming bacterium and the spore-forming bacterium does not produce a substance that substantially inhibits the growth of the spore-forming fungus; and wherein the composition is effective at increasing the yield of a plant grown from a seed to which the composition has been applied.

The agents comprising the disclosed compositions may be applied in agricultural, horticultural and seedling nursery environments. This generally includes application of agents to soil, seeds, whole plants, or plant parts (including, but not limited to, roots, tubers, stems, flowers and leaves). Microbial combinations may be used alone, however, they may additionally be formulated into conventional products such as dust, granule, microgranule, pellet, wettable powder, flowable powder, emulsion, microcapsule, oil, or aerosol. To improve or stabilize the effects of the inoculant, the agent may be blended with suitable adjuvants and then used as such or after dilution if necessary.

A worker skilled in the art would recognize that the microbial combinations may be formulated for seed treatment either as a pre-treatment for storage or sowing. The seed may form part of a pelleted composition or, alternatively, may be soaked, sprayed, dusted or fumigated with the inventive compositions. Additionally, the inventive compositions may be applied to the soil or turf, a plant, crop, or a plantation. Some areas may additionally require that an embodiment provide for slow-release materials such that the agent is designed to have an extended release period.

In use, the disclosure provides for, and includes the application of an aqueous or a non-aqueous spray composition to the crop. For example, the composition may be applied to the soil, or to a plant part (e.g., stalk, root or leaf), or both, as an aqueous spray containing spray adjuvants such as surfactants and emulsified agricultural crop oils which insure that the agent is deposited as a droplet which wets the stalk or leaf and is retained on the plant so that agent can be absorbed.

The skilled artisan would realize that the compositions disclosed herein may be applied in combination with nutrients (fertilizers) or herbicides or both, or may form part of a formulation comprising the inventive composition in combination with a fertilizer or herbicide or both. Such a formulation may be manufactured in the form of a liquid, a coating, a pellet or in any format known in the art.

The skilled artisan would realize that the compositions disclosed herein may be applied to seeds as part of stratification, desiccation, hormonal treatment, or a mechanical process to encourage germination or to terminate dormancy. Treatments including the inventive agents in combination with hormones, PEG, or varying temperature, or in combination with mechanical manipulation of the seed (i.e. piercing), are contemplated.

Further aspects of the disclosure will become apparent from consideration of the drawings and the ensuing description of further embodiments. A person skilled in the art will realize that other embodiments of the disclosure are possible and that the details of the various aspects of the disclosure can be modified in a number of respects, all without departing from the disclosure. Thus, the following drawings and description are to be regarded as illustrative in nature and not restrictive.

### BRIEF DESCRIPTION OF DRAWING

FIG. 1 shows a relationship between years in Continuous Corn and the Continuous Corn yield penalty. Adapted from Gentry et al., 2013.

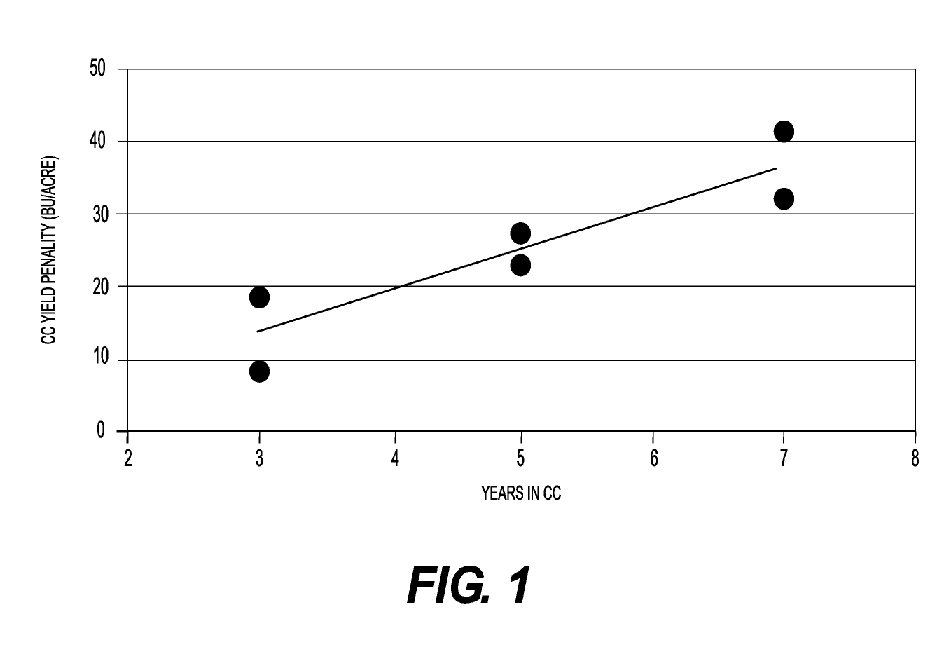

### DETAILED DESCRIPTION

In one embodiment of the disclosure, a composition comprises the fungus Trichoderma vixens isolate G1-3 (ATCC 58678) or other isolates. These microorganisms may be obtained from the American Type Culture Collection (ATCC), 12301 Parklawn Drive, Rockville, Md., 20852-1776 and other culture collections or isolated from nature.

Another embodiment comprises a composition Trichoderma (Gliocladium) virens isolate G1-21 which is being marketed under the trademark SoilGuard™. 12G by Thermo Trilogy Corporation, 9145 Guilford Road, Suite 175, Columbia, Md. 21046.

A further embodiment also comprises a composition bacterium Bacillus lentimorbus TJ 1000, which is renamed herein Bacillus amyloliquefaciens TJ1000 or 1BE, based on a more accurate determination of the name of Bacillus species that occurred before the parent patent application was filed. This microorganism was deposited with the ATTC on Oct. 31, 2001, and was assigned accession number ATCC BAA-390. Alternative embodiments of the disclosure comprise other strains which can be isolated from nature or obtained from ATCC or other culture collections.

Another embodiment comprises a composition of Bacillus subtilis var. amyloliquefaciens strain FZB24 which is being marketed under the trademark Taegro® by Earth Bioscience, Inc., 26 Sherman Court, PO Box 764, Fairfield, Conn. 06430.

A further embodiment involves combining an essentially pure culture of Trichoderma virens and an essentially pure culture of Bacillus amyloliquefaciens in a competitive culture process. The competitive culture process involves adding the Bacillus amyloliquefaciens to a growth medium about eight hours after the Trichoderma virens was added to the medium. The combined culture is then applied to a seed, for example, a corn seed. The combination grown in a competitive culture provides yield protection for seeds and plants.

A further embodiment involves growing an essentially pure culture of Trichoderma virens and an essentially pure culture of Bacillus amyloliquefaciens TJ1000 separately for five days. After the cultures are grown separately, the compositions that contain them are combined in a 50/50 combination by volume and then the combination is applied to a seed, for example, a corn seed. The combined cultures are applied to a seed provides yield protection for seeds and plants

A further step in the process involves applying either of the above combinations to a seed involves adding an aqueous solution comprising 30 grams/liter of molasses to the solution containing the combination to produce an appropriate spore count in the resulting composition. The resulting composition is then applied to the seed as a liquid mist to achieve optimum application rates per seed using the molasses as an adhesive to adhere the spores to the seed.

In a further embodiment, the bioreactor used to culture the microorganism cultures is a New Brunswick Bioflow III bioreactor. For optimal results, the agitation setting of the bioreactor is set at about 350 rpm, the aeration setting of the bioreactor is set at about 3.0 with an aeration air pressure of about 15 pounds per square inch and the temperature setting is set at about 28° C. The further growth medium for each of the individual cultures and the combined competitive culture comprises about 30 grams per liter of molasses and about 5 grams per liter of yeast extract and is referred to as a MYE medium. In a further embodiment, the medium contains about 5 milliliters of antifoam. In a further embodiment, spore production is measured by counting spores using a hemacytometer manufactured by Hausser Scientific.

A variety of seed treatments or no seed treatment may be practiced before the seed is inoculated with the disclosed inoculum. In some further embodiments, seed treatments include osmotic priming and pre-germination of the seed. Because Trichoderma vixens and Bacillus amyloliquefaciens are spore formers, the disclosed inoculum does not require high moisture levels for survival and, therefore, can be applied to seed and other materials without a sticker, such as those sold under the trade names Pelgel (LipaTech), Keltrol (Xanthan) Cellprill or Bond.

In a further embodiment, the disclosure provides for, and includes combining of a spore forming fungal strain and a spore forming bacterial strain to enhance ease of use and longevity of shelf life both as a stored product and when applied to a seed. In a further embodiment, the disclosure includes applying the disclosed Trichoderma microorganism and the disclosed Bacillus microorganism to a wettable powder, and marketing the wettable powder.

### EXAMPLES

The example is for the purpose of illustrating an embodiment and is not to be construed as a limitation.

### Example 1

### Corn-On-Corn Yield

It is well documented that planting continuous corn (corn after corn in consecutive planting seasons (non-rotated crops)) demonstrates an increasing yield penalty from year to year. For example, the study reported in Gentry et al.; Identifying Factors Controlling the Continuous Corn Yield Penalty, Agronomy Journal • Volume 105, Issue 2 • 2013 pp295-303, correlates corn-on-corn yield penalty with the number of years in continuous corn planting, and shows that the yield penalty continues to increase with each year of continuously planting of corn.

In trials a commercial formulation of QuickRoots® containing Trichoderma vixens G1-3 and Bacillus subtilis var. amyloliquefaciens TJ 1000 was applied to otherwise untreated corn seed at an application rate of 7.2 grams per 80,000 corn seed kernels. The control treatment used for comparison in each trial was untreated corn seed of the same hybrid represented in the QuickRoots® treatment. One hybrid was evaluated at each of a total of twelve field sites with a total of three distinct SmartStax® hybrids (DK53-56, DK61-16 and DK63-33) utilized across the various experimental locations to ensure appropriate relative maturities were utilized. Small plot replicated yield trials were conducted during 2014 at each of the twelve sites spanning eight states in the U.S. (MN, CO, WI [2], MI, IL [4], NE, IA and TX) utilizing standard small plot research methods and equipment. Each of these locations was planted to corn the previous cropping season and was considered corn on corn rotation sites. The experimental design was a Randomized Complete Block Design (RCBD) with two replications at each site. Corn yield data was analyzed post-harvest utilizing best linear unbiased estimation (BLUE) linear mixed model and the average yield was calculated for untreated seed and QuickRoots treated seed. Significance was determined by calculating p-values for untreated and treated conditions.

In this trial, there was a positive yield delta over the untreated control of 9.89 bu/A (p value=0.056) which was a surprising result of significantly reducing the expected corn yield penalty in the non-rotated corn fields.

Many variations of the embodiments of the present disclosure will occur to those skilled in the art. Some variations include non-competitive culturing of the inoculant organisms. Other variations call for competitive culturing. All such variations are intended to be within the scope and spirit of the disclosure.

While example systems, methods, and so on have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Additional advantages and modifications will readily appear to those skilled in the art. Therefore, the disclosure is not limited to the specific details, the representative apparatus, and illustrative examples shown and described. Thus, this application is intended to embrace alterations, modifications, and variations that fall within the scope of the appended claims. Furthermore, the preceding description is not meant to limit the scope of the disclosure. Rather, the scope of the disclosure is to be determined by the appended claims and their equivalents.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

1. A method for growing a corn plant, comprising:
selecting a field where a corn plant was grown the previous year;
planting corn seeds; and
applying a combination of Trichoderma virens and Bacillus subtilis var. amyloliquifaciens.

2. The method of claim 1, wherein said applying a combination of Trichoderma virens and Bacillus subtilis var. amyloliquifaciens is selected from the group consisting of coating said corn seed prior to planting, applying to the soil prior to planting, applying to the soil at planting, and applying to the foliage of a corn plant growing in said field.

3. A method of preventing or reducing a corn on corn yield penalty in a corn crop comprising:
applying a combination of Trichoderma virens and Bacillus subtilis var. amyloliquifaciens to said corn crop at planting;
growing said corn crop in a field that grew corn in the previous growing season.

4. (canceled)

5. The method of claim 3, wherein the yield of said corn crop is equal to or greater than the yield of the corn crop grown without the combination of Trichoderma virens and Bacillus subtilis var. amyloliquifaciens.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. The method of claim 1, where the seed is additionally coated or treated with one or more of a pesticide selected from the group consisting of a fungicide, herbicide, insecticide, acaricide and nematicide.

21. (canceled)

22. The method of claim 1, where the seed is additionally coated with a lipo-chitooligosaccharide (LCO).

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A method comprising:
planting a corn seed in soil in which corn seeds were sown the previous growing season; and
applying Trichoderma virens and Bacillus subtilis var. amyloliquifaciens to the soil, to the seed, or to a plant that germinates from the seed.

37. The method of claim 36, wherein said Trichoderma virens and Bacillus subtilis var. amyloliquifaciens is applied to the seed prior to planting.

38. The method of claim 36, wherein said applying is at least 0.25 months or more prior to planting.

39. (canceled)

40. The method of claim 36, wherein said Trichoderma virens and Bacillus subtilis var. amyloliquifaciens is applied to leaves of the plant that germinates from said seed.

41. The method of claim 36, wherein corn was sown in said soil for at least the previous two or more growing seasons.

42. (canceled)

43. The method of claim 41, wherein the yield penalty is reduced by at least about 5% or more.

44. The method of any one of claims 36, wherein one or more characteristics of plant growth such as plant height, plant weight, number of cobs, cob weight, kernel number, kernel weight, and date to maturity are enhanced by at least about 5% or more.

45. The method of claim 36, wherein the yield is enhanced by at least about 5% or more.

46. (canceled)

47. The method of claim 37, wherein said Trichoderma vixens is applied at a rate of about at least 1×10² colony forming units per seed.

48. (canceled)

49. The method of claim 37, wherein said Bacillus subtilis var. amyloliquifaciens is applied at a rate of at least 1×10² colony forming units per seed.

50. (canceled)

51. (canceled)

52. (canceled)

53. (canceled)

54. (canceled)
